(19) United States
(12) Patent Application Publication
(10) Pub. No.: US 2018/0214346 A1
(43) Pub. Date: Aug. 2, 2018

(54) PROTOCOL FOR HEALTH REHABILITATION

(71) Applicants: Robert J. Jurgensen, Raleigh, NC (US); Kathryn A. Jurgensen, Raleigh, NC (US)

(72) Inventors: Robert J. Jurgensen, Raleigh, NC (US); Kathryn A. Jurgensen, Raleigh, NC (US)

(21) Appl. No.: 15/885,376

(22) Filed: Jan. 31, 2018

Related U.S. Application Data
(60) Provisional application No. 62/453,624, filed on Feb. 2, 2017.

(51) Int. Cl. A61J 7/00; A61K 9/00; A61K 38/17; A61K 9/20; A61K 31/51; A61K 31/525; A61K 31/4406; A61K 31/4188; A61K 31/714; A61K 36/28; A61K 36/258; A61K 36/41; A61K 36/81; A61K 36/38; A61K 33/00; A61K 31/405; A61K 31/675; A61K 31/519; A61K 33/06; A61K 33/26; A61K 31/197; A61K 36/9068; A61K 36/9066; A61K 31/4045; A61K 31/375; A61K 33/30; A61K 9/50; A61K 36/886; A61K 31/122; A61K 31/593; A61K 31/20; A61K 35/741; A23L 33/105; A23L 33/135; A23L 33/15; A23L 33/16; A23L 33/12; A23L 33/175

(52) U.S. Cl. CPC A61J 7/0084; A23V 2002/00; A61K 38/1709; A61K 9/20; A61K 31/51; A61K 31/525; A61K 31/4406; A61K 31/4188; A61K 31/714; A61K 36/28; A61K 36/258; A61K 36/41; A61K 36/81; A61K 36/38; A61K 33/00; A61K 31/405; A61K 31/675; A61K 31/519; A61K 33/06; A61K 33/26; A61K 31/197; A61K 36/9068; A61K 36/9066; A61K 31/4045; A61K 31/375; A61K 33/30; A61K 9/50; A61K 36/886; A61K 31/122; A61K 31/593; A61K 31/20; A61K 35/741; A61J 7/0069; A23L 33/105; A23L 33/135; A23L 33/15; A23L 33/16; A23L 33/12; A23L 33/175; A61K 9/0095

(57) ABSTRACT

A pre-planned nutraceutical protocol for the treatment of malnutrition caused by addictive substances includes a schedule for ingesting (consuming) only non-prescription, over-the-counter ingredients. For an assembly and packaging of these ingredients, they are sequentially i) classified by type (i.e. formulation); ii) sorted by efficacy (i.e. daily requirements); and iii) assembled according to time-of-day ingestion. Once so organized, ingredient clusters can be separately packaged in holding compartments for simultaneous ingestion in accordance with a predetermined regimen.

This application claims the benefit of U.S. Provisional Patent Application Ser. No. 62/453,624, filed Feb. 2, 2017, The entire contents of Application Ser. No. 62/453,624 are hereby incorporated by reference herein,

### FIELD OF THE INVENTION

The present invention pertains to methods and systems for establishing pre-planned protocols wherein nutraceuticals are ingested and/or otherwise used to rehabilitate a person's health and wellbeing. More particularly, the present invention pertains to methods and systems for rehabilitating the health and wellbeing of a person whose physical and/or psychological condition has deteriorated or has been compromised by the use of a toxic substance(s). The present invention is particularly, but not exclusively useful for establishing pre-planned, nutraceutical protocols for rehabilitation from substance-abuse, wherein the protocols require only non-prescription, over-the-counter, ingredients.

### BACKGROUND OF THE INVENTION

For any of several reasons, a person who is using addictive substances or who has already become addicted, may want to consider self-help as a first step toward rehabilitation. The person, however, must honestly evaluate his/her situation with or without the direct assistance of trained medical personnel. With such an evaluation, a nutraceutical protocol for the individual that relies solely on non-prescription, over-the-counter, ingredients may be efficacious. And, this may be so regardless whether the protocol is to be followed or administered in a controlled program. In the event a person decides to pursue a self-help therapy, the seminal questions will inevitably concern where to start, and what steps are involved.

With a holistic approach to the task of creating an appropriate nutraceutical protocol that is effective for a rehabilitation regimen, a person is initially confronted with a plethora of selections/possibilities. The situation becomes more complex when a person acts alone, and seeks the use of only non-prescription, over products. For instance, commercially available, non-prescription nutraceutical ingredients (products) come in a variety of different forms (types), e.g. tablets, powders, capsules, sprays, lotions and patches. Moreover, each ingredient has its own unique efficacy which will dictate how it is to be used, how much can be used at a time, and how often it can be used. Knowing which ingredients to select and how to reconcile their efficacies can prove to be a daunting task.

In light of the above, it is an object of the present invention to provide a pre-planned, nutritional, health rehabilitation and recovery protocol which uses only non-prescription, over-the-counter ingredients. Another object is to provide a pre-planned, nutritional, health rehabilitation and recovery protocol that can be customized to treat malnutrition caused by the use of a specific substance(s). Still another object of the present invention is to provide a pre-planned, nutritional, health rehabilitation and recovery protocol which is easy to follow, is simple to prepare, and comparatively cost effective.

### SUMMARY OF THE INVENTION

In accordance with the present invention, a methodology for creating a nutritional rehabilitation and recovery protocol is established for an individual having an addiction or using addictive substances. Specifically, the protocol of the present invention can be followed prior to and/or during, and after a clinically controlled detoxification program. Of particular importance for the present invention is the fact that none of the ingredients required for the protocol require a prescription, and all ingredients can be purchased over-the-counter. Accordingly, the nutritional rehabilitation and recovery protocol for the present invention can be pre-planned, created, and followed, either individually or under clinical supervision.

In overview, the creation of a nutritional rehabilitation and recovery protocol first requires selecting the necessary ingredients. Depending on the nature of the addiction or addictive substance used opioids, meth, cocaine and/or alcohol), the necessary ingredients may change. In any case, however, the general methodology of the present invention is applicable.

Once the necessary ingredients for a protocol have been selected, a next step is to classify the ingredients according to their respective type (i.e. form). For example, classification can be accomplished based on a common physical characteristic of the ingredients, such as whether the ingredient is a tablet, a protein powder or a gelcap.

After classification, an important step in the methodology of the present invention is the sorting of the various ingredients into clusters. In particular, during the sorting step, ingredients having a same predetermined regimen, and thus a substantially same therapeutic efficacy, are placed in a same cluster. For example, ingredients which are to be ingested once a day are sorted into the same cluster. Likewise, ingredients which need to be ingested twice a day, or three times a day, are sorted into their respective clusters.

As envisioned for the present invention, after the sorting step has been completed, portions of the resulting clusters are assembled into suites for contemporaneous ingestion. In detail, each suite will include a portion of at least one cluster. More likely, however, each suite will include a portion from each one of several clusters. For the present invention, each assembled suite will then be simultaneously ingested according to a predetermined timetable.

As an example for the use of a timetable in accordance with the present invention, a protocol that includes ingredients which require ingestion three times a day will require a timetable having three designated times (e.g. morning [time₁], midday [time₂] and evening [time₃]). Accordingly, these ingredients will be included in three different suites. In this case, each suite will he ingested separately according to the timetable at morning [time₁], midday [time₂] and evening [time₃]. For this same protocol, other ingredients may require ingestion only twice a day. These ingredients may then be placed into any two of the suites, such as the suites intended for ingestion in the morning [time₁] and in the evening [time₃]. Further, an ingredient requiring ingestion only once a day can be incorporated into any one of the suites; for example, in the suite intended for ingestion at midday [time₂].

Packaging the ingredients of a nutritional rehabilitation and recovery protocol can be accomplished by the individual, or it can be commercially pre-packaged. For this purpose, a tray which is formed with compartments can be provided. In a preferred configuration the tray will be formed with three rows of compartments, with each row relating to a particular time in the day when the ingredients from its row are to be ingested, e.g., morning, midday, evening. Each row is then separated into seven compartments, with one compartment for each day of the week. Thus, the tray will provide three compartments for each of the seven days to create a weekly regimen for the preplanned protocol.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a functional flow chart of the sequential tasks required for creating a pre-planned nutritional rehabilitation and recovery protocol in accordance with the present invention; and

FIG. 2 is a top plan view of a packaging tray with compartments for holding suites of ingredients to be simultaneously ingested (consumed) at a same time for daily use in accordance with the pre-planned protocol.

### DESCRIPTION OF THE PREFERRED EMBODIMENTS

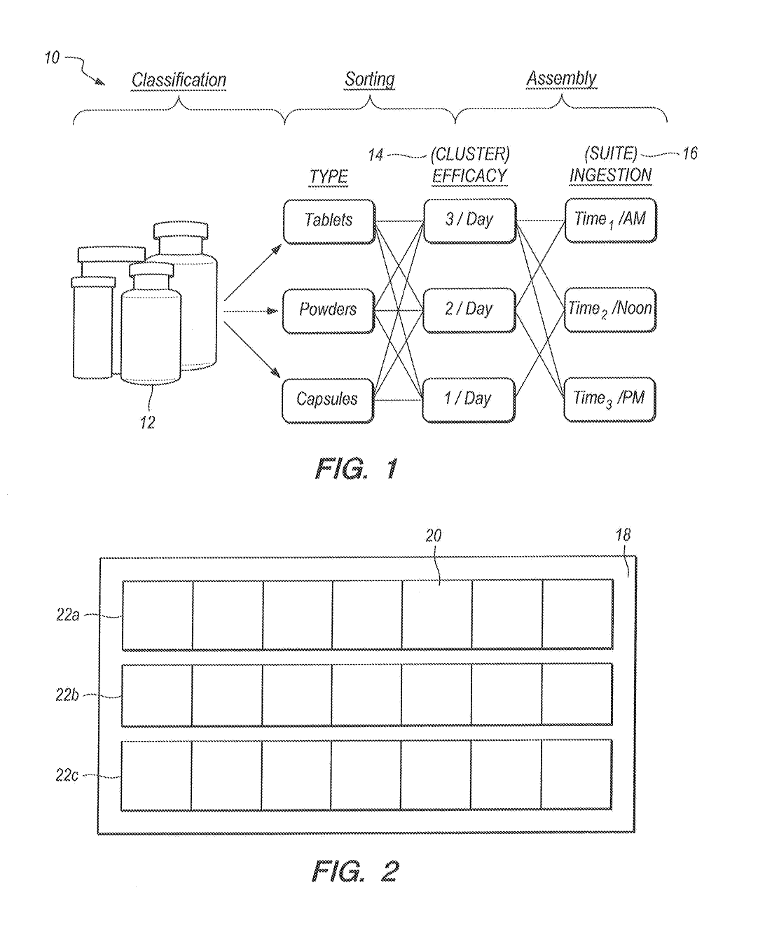

Referring initially to FIG. 1 a nutritional, rehabilitation and recovery protocol in accordance with the present invention is shown and is generally designated 10. As shown, protocol 10 is provided for the purposes of assembling and packaging a plethora of ingredients 12. Importantly, the ingredients 12 are non-prescription, over-the-counter nutraceuticals that, when properly assembled and packaged are useful for the treatment and rehabilitation of a person's health and wellbeing. More specifically, ingredients 12 can be efficacious for the recovery from use of addictive substances. Preferably, ingredients 12 are selected from the product chart provided below,

PRODUCT CHART
Nutrition Shake

| INGREDIENTS | Amt Per Day | Per Serving | Per Scoop |
|---|---|---|---|
| Caseine Protein | 40 g | 20 g | 10 g |
| Colostrum | 100 mg | 50 mg | 25 mg |
| Diosmin | 1350 mg | 675 mg | 337.5 mg |
| Hesperidin | 150 mg | 75 mg | 37.5 mg |
| L-Citruline | 40 mg | 20 mg | 10 mg |
| L-Glutamine | 200 mg | 100 mg | 50 mg |
| L-Phenylalanine | 8 g | 4 g | 2 g |
| L-Tyrosine | 1 g | 0.5 g | 0.25 g |
| Methionine | 3 g | 1.5 g | .75 g |
| Potassium | 4.7 g | 2.35 g | 1.175 g |
| Psyllium | 10 g | 5 g | 2.5 g |
| Taurine | 1.5 g | .75 g | .375 g |
| Turmeric | 1 g | 0.5 g | .25 g |

AM Vitamin Tablet

| INGREDIENTS | Amt Per Day | Per Serving | Per Tablet |
|---|---|---|---|
| B1 | 1.2 mg | 1.2 mg | 1.2 mg |
| B2 | 1.3 mg | 1.3 mg | 1.3 mg |
| B3 | 14 mg | 14 mg | 14 mg |
| B7 | 30 mcg | 30 mcg | 30 mcg |
| B12 | 2.4 mcg | 2.4 mcg | 2.4 mcg |
| Carnitine | 50 mg | 50 mg | 50 mg |
| Choline | 550 mg | 550 mg | 550 mg |
| Echinacea | 20 mg | 20 mg | 20 mg |
| Milk Thistle | 60 mg | 60 mg | 60 mg |
| Panex Ginseng | 250 mg | 250 mg | 250 mg |
| Rhodiola Rosea | 250 mg | 250 mg | 250 mg |
| Ashwaganda | 300 mg | 300 mg | 300 mg |
| St. Johns Wort | 300 mg | 300 mg | 300 mg |

PM Vitamin Tablet

| INGREDIENTS | Amt Per Day | Per Serving | Per Tablet |
|---|---|---|---|
| 5HTP | 50 mg | 50 mg | 50 mg |
| B6 | 1.3 mg | 1.3 mg | 1.3 mg |
| B9 | 133 mcg | 133 mcg | 133 mcg |
| Calcium | 100 mg | 100 mg | 100 mg |
| Fe (Iron) | 20 mg | 20 mg | 20 mg |
| GABA | 250 mg | 250 mg | 250 mg |
| Ginger | 200 mg | 200 mg | 200 mg |
| Lemon Balm | 80 mg | 80 mg | 80 mg |
| Magnesium | 400 mg | 400 mg | 400 mg |
| Melatonin | 10 mg | 10 mg | 10 mg |
| Valerian | 260 mg | 260 mg | 260 mg |
| Vitamin C | 90 mg | 90 mg | 90 mg |
| Zinc | 11 mg | 11 mg | 11 mg |

Softgel

| INGREDIENTS | Amt Per Day | Per Serving | Per Tablet |
|---|---|---|---|
| Aloe | 36 mg | 12 mg | 6 mg |
| CoQ10 | 200 mg | 66.6 mg | 33.3 mg |
| D3 | 600 IU | 200 IU | 100 IU |
| Omega 3 | 3 g | 1 g | 0.5 g |
| Omega 6 | 3 g | 1 g | 0.5 g |
| Probiotics | 3 Billion | 1 Billion | 0.5 Billion |
| | CFU's | CFU's | CFU's |

Product Chart (End)

As a first step in the protocol 10, an individual must necessarily identify the substances that have caused his/her condition. Preferably, this identification is made with the assistance of trained clinical personnel. Moreover, the assistance and supervision of trained clinical personnel may be advisable.

FIG. 1 indicates that to establish a protocol 10, the ingredients 12 need to first be classified according to their type (i.e. form). As shown in the Product Chart, the types of ingredients will depend on the respective physical characteristics of the ingredient, and will typically be obtained as tablets, protein powders, or gelcaps. It is also possible, however, that the ingredients 12 will include ointments, sprays, solutions, or patches. In any case the ingredients 12 that are selected for the protocol 10 will typically be either medicaments, proteins, vitamins, minerals, or probiotics. Although FIG. 1 shows an exemplary protocol 10 for only three different types of ingredients 12, the present invention envisions the possibility of including additional types. In any event, each ingredient 12 is handled in the same way. Importantly, each ingredient 12 needs to be an over-the-counter, non-prescription product.

After the ingredients 12 have been selected and classified, FIG. 1 indicates they need to be sorted into ingredient clusters 14 according to their respective efficacy. Preferably, sorting can be conducted according to the labeling of the particular product. Of particular import here, is the daily amount of an ingredient 12 to be ingested, and its servings/day. In FIG. 1, it will be seen that sorting results in the creation of ingredient clusters 14 of ingredient 12. As envisioned for the present invention, because each ingredient 12 may have a unique number of suggested daily servings, each ingredient cluster 14 may include several different types of the ingredients 12. Nevertheless, each ingredient cluster 14 will contain only those types of ingredients 12 that require the same number of servings per day.

Still referring to FIG. 1, individual suites 16 of different ingredient clusters 14 are then assembled according to the time of day that the particular ingredients 12 in the suite 16 are to be ingested. For example, a particular ingredient cluster 14 may require ingestion three times daily (e.g. time₁ [morning]; time₂ [midday]; and time₃ [evening]). In this case, an ingredient cluster 14 with the particular ingredients 12 will be placed in three different suites 16 (e.g. a morning suite 16, a midday suite 16 and an evening suite 16). Consequently, in accordance with the protocol 10, the individual need only ingest three different pre-planned suites 16 during a day.

With reference to FIG. 2, packaging of the protocol 10 is straight forward. For example, packaging can be accomplished using a tray 18 for holding a plurality of compartments 20. For the purpose of establishing a weekly regimen for the protocol 10, three rows 22a-c can be provided in the tray 18, with seven compartments 20 in each row 22. Thus, each compartment 20 is intended to hold a different suite 16 with the tray 18 presenting a suite 16 from three different rows 22 each day. As envisioned for the present invention, the tray 18 for a protocol 10 can be pre-packaged with assembled suites 16 and sold commercially, or it can be sold by itself for suite 16 assembly and repeated use by an individual,

While the particular pre-planned Protocol for Health Rehabilitation as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for creating a pre-planned nutritional, rehabilitation and recovery protocol which comprises the steps of:
selecting a plurality of different ingredients for creation of the protocol;
sorting individual ingredients from the plurality into different clusters according to their respective therapeutic efficacy, wherein all ingredients in a cluster have a common predetermined regimen for ingestion;
assembling a plurality of suites, wherein each suite includes at least one cluster of ingredients; and
scheduling ingestion of each suite in accordance with a predetermined timetable, wherein the timetable accounts for the regimen of individual ingredients included in the suite, and wherein all ingredients in a suite are contemporaneously ingested during the protocol.

2. The method recited in claim 1 further comprising the step of classifying the plurality of different ingredients, by type, according to respective physical characteristics of the ingredient.

3. The method recited in claim 2 wherein the physical characteristic of an ingredient is selected from the group consisting of tablets, protein powders, and gelcaps.

4. The method recited in claim 1 wherein the ingredients for the protocol are selected from the group consisting of medicaments, proteins and vitamins, minerals, arid probiotics.

5. The method recited in claim 1 wherein each ingredient is an over-the-counter, non-prescription product.

6. The method recited in claim 1 wherein the regimen for each ingredient has a suggested number of daily servings.

7. The method recited in claim 1 wherein the selecting step is predicated on determining the cause requiring implementation of the pre-planned protocol.

8. The method recited in claim 1 further comprising the steps of:
providing a tray formed with compartments; and
placing ingredients having a same time for daily use in a same compartment of the tray.

9. A pre-planned, nutritional, rehabilitation and recovery protocol which comprises:
a plurality of ingredient clusters assembled into suites for daily consumption, wherein clusters within a suite are assembled separately according to a common predetermined regimen for ingestion;
a schedule for following the predetermined regimen for ingestion wherein all ingredients in a suite are contemporaneously ingested during the protocol; and
a tray for packaging the ingredient clusters, wherein the tray has a plurality of rows and a plurality of compartments in each row, and wherein each compartment holds a suite having at least one ingredient cluster, with all ingredients in the suite having a same time for daily use.

10. The protocol recited in claim 9 wherein the ingredients are classified by type according to respective physical characteristics of the ingredient.

11. The protocol recited in claim 10 wherein the physical characteristic of an ingredient is selected from the group consisting of tablets, protein powders, and gelcaps.

12. The protocol recited in claim 11 wherein the ingredients for the protocol are selected from the group consisting of medicaments, proteins and vitamins, minerals, and probiotics.

13. The protocol recited in claim 9 wherein the ingredients are selected according to the cause requiring an implementation of the pre-planned protocol and wherein each ingredient is an over-the-counter, non-prescription product.

14. The protocol recited in claim 9 wherein the tray has three rows and each row has seven compartments to establish a weekly regimen for ingesting ingredients three times daily.

15. A pre--planned, nutritional, rehabilitation and recovery protocol which comprises:
a plurality of ingredient clusters assembled into suites for daily consumption, wherein clusters within a suite are assembled separately according to a common predetermined regimen for ingestion; and
a schedule for following the predetermined regimen for ingestion wherein all ingredients in a suite are contemporaneously ingested during the protocol.

16. The protocol recited in claim 15 further comprising a tray for packaging the ingredient clusters, wherein the tray has a plurality of rows and a plurality of compartments in each row, and wherein each compartment holds a suite having at least one ingredient cluster, with all ingredients in the suite having a same time for daily use.

17. The protocol recited in claim 16 wherein the tray has three rows and each row has seven compartments to establish a weekly regimen for ingesting ingredients three times daily.

18. The protocol recited in claim 15 wherein the ingredients are classified by type according to respective physical characteristics of the ingredient.

19. The protocol recited in claim 15 wherein the physical characteristic of an ingredient is selected from the group consisting of tablets, protein powders, and gelcaps, and wherein the ingredients for the protocol are selected from the group consisting of medicaments, proteins and vitamins, minerals, and probiotics.

20. The protocol recited in claim 19 wherein the ingredients are selected according to the cause requiring an implementation of the pre-planned protocol and wherein each ingredient is an over-the-counter, non-prescription product.
